(19) United States
(12) Patent Application Publication
(10) Pub. No.: US 2026/0131003 A1
(43) Pub. Date: May 14, 2026

(54) Method and kit for treating skin infections

(71) Applicant: Koite Health Oy, Espoo (FI)

(72) Inventors: Sakari Nikinmaa, Espoo (FI); Juha Rantala, Espoo (FI); Tommi Pätilä, Espoo (FI)

(21) Appl. No.: 19/445,860

(22) Filed: Jan. 12, 2026

Related U.S. Application Data
(63) Continuation of application No. 17/802,978, now Pat. No. 12,521,436.

(30) Foreign Application Priority Data
Feb. 27, 2020 (FI) 20205206

(51) Int. Cl. A61K 41/00; A61N 5/06

(52) U.S. Cl. CPC A61K 41/0057; A61N 5/062; A61N 5/0624; A61N 2005/0651; A61N 2005/0662

(57) ABSTRACT

Method and kit for topical treatment of a skin infection, in particular of superficial skin infections, caused by Staphylococcus aureus.The method comprises the steps of applying a photosensitizer to at least a part of the skin affected by the infection, and subjecting said part of the skin to first photons with a majority energy between 2.8 eV and 3.5 eV at a first energy level; and second photons with a majority energy between 1.24 eV and 1.65 eV at a second energy level, the ratio between the first and the second energy levels being in the range from about 10:1 to 2:1. By the method efficient treatment of Staphylococcus aureus infections can be achieved without the administration of topical antibiotics.

### CROSS-REFERENCE TO RELATED APPLICATIONS

This application is a continuation of U.S. application Ser. No. 17/802,978, filed Aug. 29, 2022, which was a National Stage Application of International Application No. PCT/FI2021/050147, filed Mar. 1, 2021, which claimed priority to Finnish Application No. 20205206, filed Feb. 27, 2020. The entirety of each application is hereby incorporated by reference.

### FIELD OF INVENTION

The present invention relates to treatment of infections, in particular skin infections. More specifically, the invention concerns a method and a kit for treating skin infections, for example superficial skin infections, caused by bacteria of the Staphylococcus genus. The invention also concerns a novel use of photosensitizers.

### BACKGROUND

Staphylococcus is a genus of Gram-positive bacteria in the family Staphylococcaceae in the order Bacillales. Despite being part of the generally harmless skin microbial flora, Staphylococcus aureus is the most dangerous of all of the staphylococcal bacteria. Skin infections are common, but the bacteria can enter the blood stream, causing potentially lethal infections. Staphylococcus aureus is the leading cause of infectious endocarditis and S. aureus bacteraemia carries a high mortality risk, with little change in mortality during history. Twenty to 60% of people are colonized by the bacteria.

Impetigo, a contagious bacterial skin disease which forms pustules and yellow crusty sores, chiefly on the face and hands, is an example of a common and generally harmless skin infection caused by bacteria of the Staphylococcus aureus or, in some cases, Streptococcus pyogenes, species. So far, Impetigo has been treated by topical application of antibiotics, such as bacitracin, often combined with netilmicin. However, allergic reactions as well as resistance formation has been proven to be a large scale problem with these antibiotics.

Resistance to antibiotics is, in fact, now considered one of the biggest threats in modern medicine. With the appearance of meticillinresistent Staphylococcus aureus (MRSA) species, new methods to reduce excessive antibiotic use, as well as means to fight resistant bacteria are urgently needed, not the least with regard to Impetigo and other superficial skin infections involving bacteria of the Staphylococcus family and in particular the Staphylococcus aureus species.

### SUMMARY OF THE INVENTION

It is an aim of the present invention to provide a method of treating infections caused by bacteria of the Staphylococcus genus, and in particular of the Staphylococcus aureus species.

It is another aim of the present invention to provide a kit for treating infections caused by bacteria of the Staphylococcus genus, and in particular of the Staphylococcus aureus species.

It is a third aim of the present invention to provide a novel use of photosensitizers in treatment of infections caused by bacteria of the Staphylococcus genus, and in particular of the Staphylococcus aureus species.

It is a fourth aim of the present invention to provide for treatment of Staphylococcus skin infections without the use of antibiotics.

The present invention is based on the finding that dual-light photodynamic therapy, i.e. photodynamic therapy carried out simultaneously using also a second wavelength, is effective against streptococcal bacteria based on the lack of catalase enzyme.

More specifically it has been found that combined antibacterial photodynamic therapy (aPDT) and antibacterial blue light (aBL) is effective against streptococcus biofilm, using a photosensitizer, in particular indocyanine green.

Thus, the present invention provides a method of treating infections, in particular skin infections, such as superficial skin infections, caused by Staphylococcus aureus in which a photosensitizer is applied to at least a portion of the skin which is affected by the infection.

The photosensitizer applied onto the skin is then subjected to first, high-energy photons and second, low-energy photons. The ratio between the energy levels of the first and the second photons is preferably 10:1 to 2:1.

The present invention also provides a sensitizer for use in the topical treatment of infections, in particular skin infections, such as superficial skin infections, caused by Staphylococcus aureus on a mammal, which sensitizer is applied to at least a part of the skin affected by the infection. The part thus treated is subsequently or simultaneously subjected to first photons with a majority energy between 2.8 eV and 3.5 eV, at a first energy level; and second photons having a majority energy between 1.24 eV and 1.65 eV, at a second energy level, the ratio between the first and the second energy levels being in the range from about 10:1 to 2:1. In particular the ratio is about 3:1.

A kit for topical treatment of skin infections, such as superficial skin infections, caused by Staphylococcus aureus on a mammal, comprises an optoelectronic device capable of and adapted to simultaneously emitting a first light consisting of high energy photons at a first energy level, and a second light consisting of low energy photons at a second energy level, the ratio between the first and the second energy levels being in the range from about 10:1 to 2:1. The kit further comprises at least one photosensitizer which can be applied on a part of the skin affected by the superficial skin infection and activated by either or both of the high energy and low energy photons.

More specifically, the present invention is mainly characterized by what is stated in the characterizing parts of the independent claims.

Considerable advantages can be obtained with the invention.

Thus, the present invention provides an efficient treatment of Staphylococcus aureus infections without, in particular entirely without, —and independently of—the administration of topical antibiotics. As stated above, antimicrobial resistance is the most important threat to modern medicine, and Staphylococcus aureus is notorious for its ability to become resistant to antibiotics. Inappropriate antibiotic use is one of the major reasons for antimicrobial resistance formation. This can be avoided by the present invention.

As a result, the treatment can be repeated an unlimited number of times without giving rise to antimicrobial resistance.

In preferred embodiments, the treatment is carried out primarily topically using non-allergic photosensitizers and by using non-coherent light, for example by using LEDs, which means that the wound area is protected against heating and burns as well as against allergic reactions.

Although the topical treatment is, as such, preferably carried out entirely without the administration of topical antibiotics, the present treatment can be combined with antiseptic topical agents.

In embodiments, the present treatment can also be combined with the use of antibiotics, such as systemic antibiotics, in particular if the treatment is initiated only at a point of time when there can be a risk of bacteria having entered the blood stream before the treatment.

The treatment can be carried out on Staphylococcus aureus infections of superficial skin tissues, for example for or during treatment of Impetigo, as well as infections of superficial skin, skin tissues, artificial skin, prosthetics, and body fluids exposed during a surgical procedure.

The treatment can be carried out on infections generally caused by Staphylococcus aureus strains, including infections caused by methicillin resistant Staphylococcus aureus (MRSA).

The treatment can be carried out generally on mammals, including humans and animals.

Next, embodiments will be examined with the aid of a detailed description and experimental results.

### BRIEF DESCRIPTION OF THE DRAWINGS

FIG. 1 is a bar chart showing the efficiency of various light treatments on Staphylococcus aureus growth medium suspensions in terms of the count of colony-forming units after a one-day treatment with single-dose light exposure;

FIG. 2 is a bar chart showing the corresponding efficiency as FIG. 1 after three-day incubation;

FIG. 3 is a bar chart showing of various light treatments on Staphylococcus aureus growth medium suspensions in terms of the count of colony-forming units after three-day incubation with repeated daily dosage; and

FIG. 4 is a bar chart showing the corresponding efficiency as in FIG. 3 but after six-day incubation with repeated daily dosage.

### EMBODIMENTS

### Definitions

In the present context, “photodynamic therapy”, also referred to by the abbreviation “PDT”, stands for any therapy where light is converted to some form of reactive oxygen. Examples of reactive oxygen includes singlet oxygen, oxygen radicals and oxygen ions.

“Antimicrobial photodynamic therapy”, also referred to by the abbreviation “aPDT”, is a photochemistry-based method that uses photons to activate “sensitizers” that, in the activated state, impart antimicrobial, in particular antibacterial, effect.

“Antimicrobial blue light”, also referred to by the abbreviation “aBL”, is light, typically in the wave length area of 400 nm to 470 nm, such as 400 nm to 430 nm or 405 to 470 nm, or 405 to 430 nm, or about 405 nm, that exhibits for example intrinsic antimicrobial effect without the involvement of exogenous photosensitizers.

“Photosensitizers” are compounds or molecules that are capable of absorbing electromagnetic radiation for example in the ultraviolet or visible region and transferring it to adjacent molecules. Typically, the photosensitizers have de-localized π systems.

Although the term “photosensitizer” at instances is used in the singular voice in the present context, the term also comprises several compounds or molecules. In particular, it comprises mixtures or sequential use of two or more photosensitizer compounds or molecules. In case of using a plurality of photosensitizer compounds or molecules, at least one is suitable for use with high energy photons and at least one is suitable for use with low energy photons.

In one embodiment, one photosensitizer selected is capable of achieving endogenous antimicrobial action in combination with the corresponding light, and one photosensitizer is selected which is capable of achieving exogenous antimicrobial action in combination with the corresponding light.

The photosensitizers can be naturally occurring compounds (“natural photosensitizers”) and synthetic compounds. Indocynine Green is a particularly preferred photosensitizer.

“Non-coherent” when used in connection to light means that the amplitude and phase of the emitted light waves fluctuate randomly in space and time. One embodiment comprises using LEDs as non-coherent light sources. Another embodiment comprises using UVC lamps as non-coherent light sources.

“High energy photons” are photons with energy in the range from 3.5 eV to 2.8 eV, in particular about 3.2 to 2.9 eV or 3.17 to 2.95 eV. Typically, such photons are contained in light having a wavelength in the range of about 350-450 nm, for example about 380 to 430 nm, such as 390 to 410 nm, or about 405 nm.

“Low energy photons” are photons with energy in the range from 1.24 eV to 2.48 eV, in particular 1.3 to 2.4 eV, for example 1.4 to 1.6 eV or 1.45 to 1.56 eV. Typically, such photons are contained in light having a wavelength in the range of about 500 to 1000 nm, for example about 780 to 830 nm, for example about 810 nm.

Light with photons having “a majority energy in the range from 3.5 eV to 2.8 eV” stands for light, for example in the form of a light beam or light ray, in which at least 50%, in particular at least 60% or at least 70% or at least 80% or at least 90% or at least 95%, of the photons—as indicated by their energy—have an energy in the range from 3.5 eV to 2.8 eV, or 3.2 eV to 2.9 eV, such as 3.17 to 2.95 eV.

Light with photons having “a majority energy in the range from 1.24 eV to 2.48 eV” stands for light, for example in the form of a light beam or light ray, in which at least 50 %, in particular at least 60% or at least 70% or at least 80% or at least 90% or at least 95%, of the photons—as indicated by their energy (or wavelength)—have an energy in the range from 1.24 eV to 2.48 eV or 1.3 eV to 2.4 eV, such as 1.4 to 1.6 eV or 1.45 to 1.56 eV.

Generally, the term “majority energy” means that more than 50%, in particular more than 60%, for example more than 70% or more than 80% of the energy of the light lies in the indicated range.

Generally speaking, it has been found that dosing, in particular simultaneously, dosing of both high and low energy photons, in particular together with a low energy photon activated photosensitizer, increases the antibacterial effect of light compared to dosing of either group of photons separately.

A first embodiment comprises a method of treating skin infections caused by Staphylococcus aureus comprising the steps of
applying a photosensitizer to at least a part—preferably a majority, or all—of the skin affected by the infection, and
subjecting said part of the skin to first photons with a majority energy between 2.8 eV and 3.5 eV, at a first energy level; and second photons with a majority energy between 1.24 eV and 1.65 eV, at a second energy level. As will appear from the experimental data given below, dual-light aPDT is efficient against Staphylococcus aureus biofilm. The simultaneous, synchronized application of a photosensitizer, such as ICG combined with low-energy light (e.g. about 810 nm light, i.e. aPDT) and about 405-nm aBL, i.e. high-energy light results in a significantly improved antibacterial efficacy, when compared to single-light aPDT or aBL. Interestingly, the antibacterial effect was greatest, when the relative amount of aBL was increased to two thirds or more of the total amount of light irradiance.

Thus, in one embodiment, the ratio between the first and the second energy levels is in the range from about 10:1 to 2:1, in particular from about 5:0.7 to 3.5:0.8, for example 3.1:0.9, such as about 3:1.

In one embodiment, during the application of the photosensitizer, at least 50%, in particular at least 60%, for example at least 70%, such as at least 80%, preferably at least 90% of the infected area is covered with the photosensitizer. The photosensitizer can be applied onto the whole infected area and further such that at least some of the non-infected area adjacent to the infected area is covered as well.

In a first embodiment, the present technology provides for a method and kit of treating biological surfaces with electromagnetic radiation in the form of light of two different energy levels, a first light with photons having a majority energy in the range from 3.5 eV to 2.8 eV and a second light with photons having a majority energy in the range from 1.24 eV to 2.48 eV.

The treatment is carried out by simultaneously directing the photons of the first light and the second light against the part of the skin affected by the bacterial infection.

In one embodiment the photons have at least 50% of their energy at 3.17 eV to 2.95 eV and 1.56 eV to 1.45 eV, respectively. In one embodiment, the photons have at least 50% of their energy in a range corresponding to the wavelength of about 390 to 420 nm and 795 to 855 nm, respectively, for example about 405 nm and 810 nm, respectively.

In one preferred embodiment, the light employed is non-coherent. Such light is in particular obtained by using Light Emitting Diodes (LEDs).

In one embodiment,
non-coherent radiant light energy is generated at least two different energy levels, a first and a second energy level;
from the non-coherent radiant light energy there is provided first light having a wavelength corresponding to the majority energy of the first energy level, and second light having a wavelength corresponding to the majority energy of the second energy level; and
the first and second light is then simultaneously directed against the part of the skin which has been treated with the photosensitizer and affected by the bacterial infection.

In one embodiment, the light is generated using an optoelectronic component and device thereof (in particular in a kit), which is capable of and, thus, adapted to simultaneously emitting a first light consisting of high energy and a second light consisting of low energy photons, said first and said second light amounting to at least 80% of all light emitted from the optoelectronic component or device.

In one embodiment, a method according to any of the above embodiments is carried out using an anti-microbial optoelectronic component and device thereof, such as an LED light source, by simultaneously emitting high energy photons absorbed by endogenous molecules and low energy photons absorbed by exogenous molecules.

In the method, the sensitizer for use in the topical treatment of a superficial skin infection caused by Staphylococcus aureus is applied to a part of skin affected by the infection, and that part is subsequently or simultaneously subjected to first photons with a majority energy between 2.8 eV and 3.5 eV at a first energy level; and second photons with a majority energy between 1.24 eV and 1.65 eV at a second energy level, the ratio between the first and the second energy levels being in the range from about 10:1 to 2:1.

In one embodiment, the ratio between the first and the second energy levels is 5:0.8 to 3.5:0.9.

The sensitizer can be selected from the group of natural compounds comprising hypericin, curcumin, phenalenone derivatives, Cercosporin, psoralen, xanthotoxin, Angelicin, alpha-Terthienyl, Phenylthepatriyne, THC, Cannabidiol (CBD) and combinations thereof, and the group of synthetic photosensitizers comprising RB (Rose Bengal), MB, Porphyrin derivatives, Curcumin derivatives, Methylene Blue, Indocyanine Green, Erythosine, Phenalenone derivatives, Fullerene derivatives, Xanthene derivates, resveratrol, berry extracts, such as lingonberry and blueberry including polyphenolic compounds and/or anthocyanine compounds and combinations thereof, or the photosensitizer is a the combination of at least one photosensitizer is selected from the group of natural compounds listed above and at least one photosensitizer is selected from the group of synthetic compounds listed above.

In one embodiment, the sensitizer used in the topical treatment of an infection is Indocyanine Green.

The sensitizer for use in the topical treatment is subjected to light energy, in particular non-coherent radiant light energy, at a first wavelength from 400 to 430 nm at first power density, and at a second wavelength from 780 to 830 nm at a second power density, the ratio of the power density of the light energy of the first wavelength to the power density of the light energy of the second wavelength being 5:0.8 to 3.5:0.9, in particular about 3:1.

In one embodiment, the non-coherent radiant light energy has a first wavelength of 400 to 430 nm, for example 405 nm, preferably at a dosage of 1 to 750 J/cm², for example 5 to 600 J/cm², in particular 10 to 250 J/cm² and preferably a power density of from about 10 to about 2500 mW/cm². The radiant light energy has further a second wavelength of 780 to 830 nm, for example 810 nm, preferably at a dosage of 1 to 750 J/cm², for example 5 to 600 J/cm², in particular 10 to 250 J/cm² and a power density of from about 10 to about 2500 mW/cm².

In one embodiment, the dosage of the radiant light energy having a first and a second wavelength, respectively, is in the range of 1 to 600 J/cm² (for each light).

In one embodiment, the dosage of the radiant light energy having a first and a second wavelength, respectively, is in the range of 1 to 120 J/cm² (for each light).

In one embodiment, the dosage of the radiant light energy having a first and a second wavelength, respectively, is in the range of 1 to 10 J/cm² (for each light) to achieve a bacteriostatic effect.

In one embodiment, the dosage of the radiant light energy having a first and a second wavelength, respectively, is in the range of more than 120 J/cm² and up to about 250 J/cm² (for each light).

The topical treatment of a skin infection caused by Staphylococcus aureus is generally carried out, non-intermittently, for a period of time from 0.5 s to 120 min.

In one embodiment, the topical treatment, which preferably has a duration of 0.5 s to 120 min, is carried out 1 to 5 times a day, for 1 to 14 days, in particular 1 to 7 days.

In one embodiment, the topical treatment, which preferably has a duration of 0.5 s to 120 min, is repeated 2 to 50 times during 1 to 14 days. In one embodiment, the topical treatment is repeated 2 to 25 times during 1 to 7 days.

The photo-sensitizer is typically applied, for example in liquid form or as a gel, in amounts of about 0.01 mg/ml to 10 g/ml, for example 0.1 mg/ml to 1 g/ml.

In some embodiments, the photo-sensitizer is applied to the superficial skin infection at 0.0001% (w/v) to 1% (w/v).

In an embodiment, a kit for topical treatment of a superficial skin infection caused by Staphylococcus aureus on a mammal, comprises an optoelectronic device capable of (and adapted to) simultaneously emitting a first light consisting of high energy photons at a first energy level, a second light consisting of low energy photons at a second energy level, the ratio between the first and the second energy levels being in the range from about 10:1 to 2:1, for example 5:0.8 to 3.5:0.9, in particular about 3:1, and at least one photosensitizer which can be activated by at least either of the high energy and low energy photons.

One embodiment provides a kit with an optoelectronic device is capable of (and adapted to) emitting light, in particular non-coherent light, comprising
first photons with a majority energy between 2.8 eV and 3.5 eV at a first energy level, or at a first wavelength from 400 to 430 nm, at a dosage of 1 to 750 J/cm², and a power density of from about 10 to about 2500 mW/cm² for a period of time from 0.5 s to 120 min, and
second photons with a majority energy between 1.24 eV and 1.65 eV at a second energy level, or at a second wavelength from 780 to 830 nm, at a dosage of 1 to 750 J/cm², and with a power density of from about 10 to about 2500 mW/cm² for a period of time from 0.5 s to 120 min.

In one embodiment, a kit comprising an optoelectronic device is adapted to emitting two wavelengths simultaneously or at a time interval of 0.001 to 1000 ms, for example 0.1 to 500 ms or 10 to 250 ms, such as about 100 ms, from each other, together with a photosensitizer or a plurality of photosensitizers, and it is typically adapted to carrying out a method of topical treatment of a skin infection on the skin of a mammal caused by Staphylococcus aureus.

Dual-light aPDT is effective against Staphylococcus aureus biofilm and provides a sustained effect. Increase in the relative ratio of aBL strengthens the antibacterial effect, especially when treatment is repeatedly applied. Dual-light aPDT therefore provides effective treatment of Staphylococcus aureus infections and eliminates the need for use of antibiotics in topical antibacterial applications.

In one embodiment, the Staphylococcus aureus infection comprises infections on the skin, such as superficial skin infections, for example Impetigo. Also abscesses can be treated.

In one embodiment, the Staphylococcus aureus infection comprises an infection of tissues, such as organ tissues, superficial skin, skin tissues and body fluids during a medical procedure, such as tissues, organs, prosthetics and body fluids exposed during a medical procedure, such as a surgical procedure. One embodiment comprises treatment of infected organ tissues of, for example, joints, heart and lung. One embodiment comprises treatment of prosthetic parts or artificial parts, such as prosthetics, for example heart valves or prosthetic joints, or artificial skin, as well as adjacent tissue and body fluids, exhibiting infections.

In one embodiment, biofilms exhibiting Staphylococcus aureus infections are treated.

The above embodiments can be carried out on humans as well as on animals.

The following examples illustrate embodiments.

### Materials and Methods

Monospecies S. Aureus biofilm model experiments were performed to study the effect of single, or recurring application of dual-light aPDT or its parts during the biofilm formation process. Flat-bottom Nunclon Delta well plates (Thermo Fisher Scientific Inc, US), were used, and the biofilm experiments were divided into different setups based on maturation age and the therapy given.

### Study Protocols

Applications of both single- and dual-light therapies were scheduled to mimic the daily use of antibacterial light therapy. The total amount of light irradiance as kept unchanged in different treatment protocols, and the concentration of the ICG photosensitizer (if given) were identical. In the first setting, S. aureus biofilm was incubated for either one day or three days, and a single dose of light with ICG was applied at the end of the growth period. In the second setting, the biofilm was incubated for either three days or six days, and a daily dose of light ICG with was applied during incubation. In each setting, the last treatment was followed by plating onto brain heart infusion (BHI)-agar dishes for colony-forming unit (CFU) counting.

### Biofilm Model

Staphylococcus Aureus bacteria were grown for 18 h in an incubator (NuAire DH autoflow 5500, NuAire inc, US), at +36 degrees C, 5% CO₂ in BHI broth (Bio-Rad 3564014, Bio-Rad Laboratories, Inc, US). The resulting bacterial suspension was diluted with a 0.9% NaCl solution until an optical density (OD) of 0.46 was reached. The optical density was measured by a spectrophotometer (Varian Cary 100 Bio UV-VIS, Agilent Technologies, Inc, US), and then with a Den 1 McFarland Densitometer (Biosan, Riga, Latvia).

Biofilms were grown in flat-bottom 96-well plates (Thermo Fisher Scientific Inc, US) by placing 100 μl of 0.46 OD S. aureus suspension in each well, with 100 μl of BHI-broth growth medium. The well plates were then incubated in a growth chamber (36° C., 5% CO²). The BHI-broth medium was changed daily to supply fresh growth medium and to wash away the debris. The change of the medium in each well was performed by removing 100 μl of the medium and replacing it with a similar amount of fresh BHI broth.

### Light Exposure

Before the light exposure took place, the growth medium was meticulously removed by pipetting and subsequently replaced with an equal amount of indocyanine green solution (Verdye, Diagnostic Green, GmBH), tittered to a concentration of 250 μg/ml. The indocyanine green was left to incubate at room temperature and in the dark for 10 minutes. After this incubation period, the biofilm was washed with a 0.9% NaCl solution. Then, the 0.9% solution of NaCl was added to each well to reach a total volume of 200 μl.

Light exposure was performed using specific, custom-made LED light sources (Lumichip Oy, Espoo, Finland). The exposure time was calculated from the determined light amount and known irradiances, which had been previously measured with a light energy meter (Thorlabs PM 100D with S121C sensor head, Thorlabs Inc, US) and a spectroradiometer (BTS256, Gigahertz-Optik GmbH, Germany), respectively. After the exposure, the BHI broth was changed and the plates were placed in the incubator, or, if the light exposure was final, the biofilm was scraped for CFU counting, as described below. Excitation lights were applied with two single-wave LED light sources with peak intensities at 810 nm or at 405 nm, and with a dual-wave LED light chip simultaneously producing two separate peak intensities at 405 nm and at 810 nm.

Antibacterial photodynamic therapy light exposure was administered at an 810-nm peak wavelength LED array on top of the well plate. The resulting light irradiance was 100 mW/cm², and the provided light energy was 100 J/cm². Antibacterial blue light was administered at a 405-nm peak wavelength LED array, with a resulting irradiance of 80 mW/cm², and resulting light energy of 100 J/cm². The dual light was administered with two light peaks identically placed and providing LED arrays on top of the well plate, producing a synchronous irradiance of 50 mW/cm² for the 405-nm light, and 50 mW/cm² for the 810-nm light. The light energies produced were 50 J/cm² (405 nm) and 50 J/cm² (810 nm), respectively. To rule out an LED well-heating effect on bacterial viability, temperature controls were measured (Omega HH41 Digital Thermometer, Omega Engineering, US) in the biofilm wells to confirm temperature levels below 35 degrees during the treatment, with a 100 mW/cm² radiant flux.

The antibacterial efficacy of dual-light treatment was also tested in terms of the relative amounts of 810-nm and 405-nm light irradiance. Three different light combinations were employed, with a simultaneous use of the single-peak-emitting light sources. Firstly, a 1:1 irradiance ratio of aBL to aPDT was applied, with 70 mW/cm² irradiance for the 405-nm light and 70 mW/cm² for the 810-nm light, the light energy emitted being at 50 J/cm² and at 50 J/cm², respectively. Secondly, a 3:1 irradiance ratio of aBL to aPDT was applied, with 130 mW/cm² irradiance for the 405 nm light and 40 mW/cm² for the 810 nm light, the light energy provided being at 75 J/cm² and at 25 J/cm², respectively. Thirdly, a 1:3 irradiance ratio of aBLto aPDT was applied, with 40 mW/cm² irradiance for the 405-nm light and 130 mW/cm² for the 810 nm light, the light energy applied being at 25 J/cm² and at 75 J/cm², respectively.

### Colony-Forming-Unit Counting

Subsequent to the final light exposure of each experiment, the entire biofilm was removed from the well by collecting the broth together with the dense biofilm. The biofilm was then placed into a 1-ml test tube, forming 200 μl of suspension. After meticulous vortexing (Vortex Genie, Scientific Industries Inc, US), serial dilutions from 1:1 to 1:100 000 were done, using sterile ART filter tips (Thermo Scientific, Waltham, US). To enumerate the viable cells, 100 μl of resulting biofilm dilution was then evenly spread over an entire BHI plate, using a sterile L-shape rod.

The plates were then assembled into the incubator, the bacteria were grown for 48 h, and the plates were photographed (Canon D5 DSLR camera with Canon EF 24-70 mm f/4 L lens, Canon, Japan) on a light table (Artgraph Light Pad Revolution 80, Artograph Inc, US). Colony-forming units were assessed with Image J software (National Institute of Health, US). Typically, numbers between 30 and 800 were considered to be in the range where the data was statistically most reliable, and the number of bacterial colonies was calculated accordingly.

### Statistical Analysis

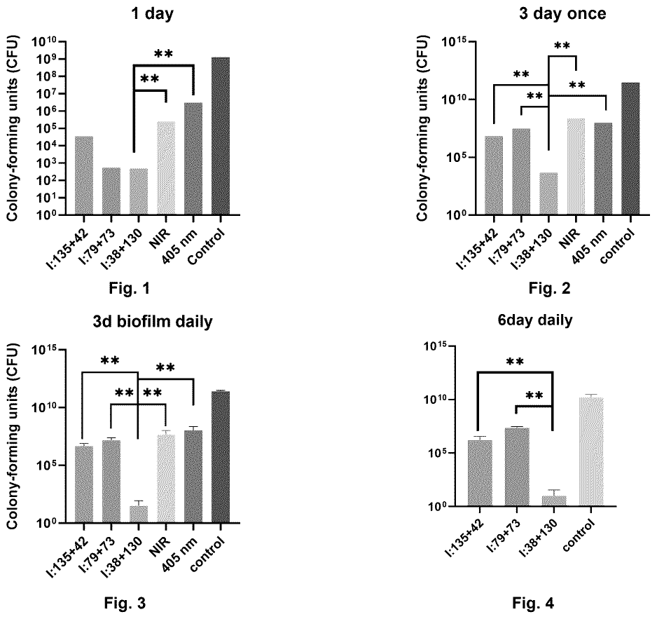

Colony-forming-unit counts were compared with the non-parametric Mann-Whitney U-test, using GraphPad Prism 8 software (GraphPad Software, San Diego, US). Results FIGS. 1 to 4 illustrate the results obtained by the tests. In the drawings, the six bars stand for the following (from left to right): first column: 1:3 aBL/aPDT ratio; second column: 1:1 aBL/aPDT ratio; third column: 3:1 aBL/aPDT ratio; fourth column: aPDT (810 nm); fift column: 405 nm; and sixth column: control. As will appear, on a one-day biofilm, dual-light aPDT was significantly more efficient than single-light aBL or aPDT, although all modalities were bactericidal. On a three-day maturated biofilm, a single exposure of aPDT or dual-light aPDT was more efficient than aBL, resulting in a four logarithmic scale reduction in bacterial counts. Surprisingly, a significant difference was observed between different dual-light relative energy rations where 1/4 NIR to 3/4 aBL was most effective. The 1/4 NIR and 3/4 aBL ratio increased with antibacterial effectivity compared to 1/1 and 3/4 NIR and 1/4 aBL relative energy rations. The largest difference was observed in 6-day daily treated biofilm model. This study demonstrates the efficacy of dual-light aPDT against Staphylococcus aureus biofilm. The simultaneous, synchronized application of an ICG/810-nm aPDT and 405-nm aBL resulted in a significantly improved antibacterial efficacy, when compared to single-light aPDT or aBL. Interestingly, the antibacterial effect was greatest, when the relative amount of aBL was increased up to two thirds of the total amount of light irradiance. Most

importantly, the antibacterial effect

1. A method for treatment of a surface comprising (i) bacteria of the genus Staphylococcus or (ii) an infection caused by the bacteria of the genus Staphylococcus, the method comprising:
applying a photosensitizer to at least a portion of the surface; and
subjecting the surface with the applied photosensitizer to first light comprising first photons at a first energy level and to second light comprising second photons at a second energy level, the ratio between the first energy level and the second energy level being in the range from about 10:1 to about 2:1.

2. The method according to claim 1, wherein the first photons have a majority energy between 2.8 eV and 3.5 eV, and wherein the second photons have a majority energy between 1.24 eV and 1.65 eV.

3. The method according to claim 2, wherein:
non-coherent radiant light energy is generated at the first energy level and the second energy level; and
from the non-coherent radiant light energy, there is provided the first light, the first light having a wavelength corresponding to the majority energy of the first energy level, and the second light, the second light having a wavelength corresponding to the majority energy of the second energy level.

4. The method of claim 1, wherein the first light and the second light are simultaneously directed against the surface.

5. The method according to claim 1, wherein the bacteria of the genus Staphylococcus comprise Staphylococcus aureus.

6. The method according to claim 1, wherein the surface comprises a biological surface.

7. The method according to claim 1, wherein the surface comprises a biofilm comprising the bacteria of the genus Staphylococcus.

8. The method according to claim 1, wherein the method treats superficial skin infections caused by Staphylococcus aureus.

9. The method according to claim 1, wherein the method treats infections caused by Staphylococcus aureus on or in organs, organ tissues, superficial skin, skin tissues, body fluids, prosthetics and parts thereof, or artificial tissues.

10. The method of claim 1, wherein the photosensitizer is applied to the skin of a mammal and the skin is subjected to the first light and the second light.

11. The method according to claim 1, wherein the first light and the second light are emitted from an optoelectronic device having a plurality of semiconductor chips that are connected in series or in parallel.

12. The method according to claim 1, wherein the photosensitizer is selected from a natural compound, a synthetic photosensitizer, or combinations thereof,
wherein the natural compound is selected from the group consisting of hypericin, curcumin, phenalenone derivatives, cercosporin, psoralen, xanthotoxin, angelicin, alpha-terthienyl, phenylthepatriyne, THC, cannabidiol (CBD), and combinations thereof, and
wherein the synthetic photosensitizer is selected from the group consisting of RB (Rose Bengal), porphyrin derivatives, curcumin derivatives, methylene blue, indocyanine green, erythosine, phenalenone derivatives, fullerene derivatives, xanthene derivatives, and combinations thereof.

13. The method according to claim 12, wherein the photosensitizer is Indocyanine Green.

14. The method according to claim 1, wherein the first and second photons are produced by one or more light-emitting diodes.

15. The method according to claim 1, wherein the first light is directed against the surface at a dosage of 1 to 750 J/cm² and with a power density of from about 10 to about 2500 mW/cm² for a period of time from 0.5 s to 120 min, and the second light is directed against the surface at a dosage of 1 to 750 J/cm² and with a power density of from about 10 to about 2500 mW/cm² for a period of time from 0.5 s to 120 min.

16. The method according to claim 1, wherein the treatment is carried out for a duration of 0.5 s to 120 min, 1 to 5 times a day, for 1 to 14 days.

17. A method for treatment of a surface comprising (i) bacteria of the genus Staphylococcus or (ii) an infection caused by the bacteria of the genus Staphylococcus, the method comprising:
applying a photosensitizer to at least a portion of the surface; and
subjecting said part of the surface with the applied photosensitizer to first photons with a majority energy between 2.8 eV and 3.5 eV and second photons with a majority energy between 1.24 eV and 1.65 eV.

18. The method of claim 17, wherein the first photons have a first energy level and the second photons have a second energy level, the ratio between the first energy level and the second energy level being in the range from about 10:1 to about 2:1.

19. A kit for treatment of a surface comprising (i) bacteria of the genus Staphylococcus or (ii) an infection caused by the bacteria of the genus Staphylococcus, the method comprising:
an optoelectronic device configured for emitting a first light at a first energy level and a second light comprising second photons at a second energy level, the ratio between the first and the second energy levels being in the range from about 10:1 to 2:1, and
at least one photosensitizer which can be activated by either or both of the high energy and low energy photons.

20. The kit according to claim 19, wherein the first photons have a majority energy between 2.8 eV and 3.5 eV, wherein the second photons have a majority energy between 1.24 eV and 1.65 eV, and wherein the optoelectronic device is configured for simultaneously emitting the first light and the second light.
